(19) United States
(12) Patent Application Publication
(10) Pub. No.: US 2002/0150579 A1
(43) Pub. Date: Oct. 17, 2002

(54) B lymphocyte stimulator (BLyS) as a marker in management of systemic lupus erythematosus

(76) Inventors: Robert P. Kimberly, Birmingham, AL (US); Tong Zhou, Birmingham, AL (US)

Correspondence Address: Glenna Hendricks, Esq., P.O. Box 2509, Fairfax, VA, 22031-2509, US

(21) Appl. No.: 10/041,663

(22) Filed: Jan. 10, 2002

Related U.S. Application Data
(60) Provisional application No. 60/260,823, filed on Jan. 10, 2001.

(51) Int. Cl. A61K 39/395; A61K 9/127; G01N 33/53

(52) U.S. Cl. 424/146.1; 424/450; 435/7.1

(57) ABSTRACT

The instant invention provides means for evaluating onset of and progress of autoimmune diseases characterized by presence of autoantibodies against nuclear antigens. The invention evaluates the level of BLyS using antibodies bond to BLyS as means for evaluating the onset and progress of disease conditions. The method provides for evaluating self-antigen driven antibody related disease my measuring the level of BLyS in the blood using BLyS-binding antibodies by:

1) exposing samples of (a) sera or plasma from patients suffering from diseases arising from self-antigen driven autoimmune response to anti-BLyS antibody and (b) at least one control sample containing a known amount of BLyS to anti-BLyS antibodies,

2) incubating the product of step 1 for a time sufficient to allow said anti-BLyS antibody to bind to BLyS in the samples,

3) evaluating the amount of anti-BLyS antibody bound to BLyS in each of the patient and control samples, and

4) comparing the amount of BLyS in control samples patient samples to determine amount of BLyS in the patient samples.

The method can be used both as a diagnostic and as a means of monitoring disease progression and response to treatment.

This application takes priority from Provisional Patent Application 60/260,823, filed Jan. 11, 2001.

The work resulting in this invention was partially supported by the U.S. Government through the National Institutes of Health. Therefore, the U.S. Government has certain rights in this invention.

### FIELD OF THE INVENTION

This invention relates to the use of B Lymphocyte Stimulator (BLyS) as a means of evaluating the presence and progress of disease conditions such as systemic lupus erythematosus (SLE).

### BACKGROUND OF THE INVENTION

The tumor necrosis factor (TNF) superfamily plays a crucial role in regulation of immune response by inducing either apoptosis or proliferation or both in lymphocytes. B lymphocyte stimulator (BLyS) (also known as BFF, TALL-1, THANK, zTNF4), a newly identified member in the TNF gene family, is a type II membrane protein which exists in both membrane-bound and soluble forms. BLyS exhibits a strong co-stimulatory function for B cell activation in vitro. Systemic administration of soluble BLyS results in B cell expansion and elevated levels of immunoglobulins. More importantly, it has been recently demonstrated that BLyS transgenic mice develop severe B cell hyperplasia and auto-immune lupus-like disease characterized by the presence of autoantibody against nuclear antigens and immune complex deposits in the kidney.

Human systemic lupus (SLE) is a systemic autoimmune disease characterized by autoantibody production against self antigens. Autoreactive B cells are driven by self antigen, but the factors that promote the loss of B cell tolerance and drive autoantibody production are still unknown. Endogenous B cell stimulatory factors are attractive candidates in this process, and BLyS has been recently identified as a potent B cell stimulatory molecule associated with systemic autoimmune disease in animals. In two murine models of SLE, MRL/lpr and NZB/WF1 mice, there are increased serum levels of BLyS which seem to correlate with autoimmune kidney damage. The treatment with the soluble BLyS receptor significantly improved the survival of mice with lupus.

### SUMMARY OF THE INVENTION

The instant invention provides means for evaluating onset of and progress of autoimmune diseases characterized by presence of autoantibodies against nuclear antigens. The invention evaluates the level of BLyS using antibodies bond to BLyS as means for evaluating the onset and progress of disease conditions. The method provides for evaluating self-antigen driven antibody related disease my measuring the level of BLyS in the blood using BLyS-binding antibodies by:

1) exposing samples of (a) sera or plasma from patients suffering from diseases arising from self-antigen driven autoimmune response to anti-BLyS antibody and (b) at least one control sample containing a known amount of BLyS to anti-BLyS antibodies,

2) incubating the product of step 1 for a time sufficient to allow said anti-BLyS antibody to bind to BLyS in the samples,

3) evaluating the amount of anti-BLyS antibody bound to BLyS in each of the patient and control samples, and

4) comparing the amount of BLyS in control samples patient samples to determine amount of BLyS in the patient samples.

The method can be used both as a diagnostic and as a means of monitoring disease progression and response to treatment.

### DETAILED DESCRIPTION OF THE INVENTION

The instant invention arose from a study of serum levels and function of BLyS in patients with SLE. Results demonstrate that BLyS found in sera of SLE patients functions as a stimulator for B cell activation and is markedly elevated compared to normal controls. While it was known that BLyS was associated with systemic autoimmunity in animal models of spontaneous autoimmune disease, it had not been previously recognized and demonstrated that BLyS could be used as a marker for evaluation of presence and progress of diseases associated with self-antigen driven autoimmune diseases such as systemic lupus erythematosus (SLE) and Sjogren's syndrome (SS). It was found that increased BLyS in such patients is associated with higher levels of anti-dsDNA antibody of the IgG, IgM and IgA classes and with higher levels of total serum IgA, which suggests that the role for BLyS might be to selectively trigger B cell tolerance loss driven by dsDNA. However, BLyS was not associated with elevated anti-Sm and anti-SmRNP (two major anti-nuclear protein autoantibodies) levels or with elevated total IgG and IgM levels. The discovery of these relationships indicated that BLyS can be an indicator for early activation of autoimmune diathesis and evidences a critical role in triggering activation of self-antigen driven autoimmune cells in human autoimmune diseases such as human SLE. The discovery also suggests BLyS as a target in treating systemic autoimmunity.

### Materials and Methods

Human Subjects:

Peripheral blood was obtained from 150 SLE patients meeting the American College of Rheumatology (ACR) criteria for the classification of disease. Serum from 40 patients was harvested and stored at −30° to −70° C. until use, and plasma from a second, independent cohort of 110 SLE patients was collected and also stored at 30° C. to 70° C. until use. Two additional subgroups of patients with positive antinuclear antibody (ANA) titers, but who did not meet the ACR criteria were also selected. Disease activity was assessed by direct clinical assessment and the Systemic Lupus Activity Measure (SLAM), usually on the same day as drawing the blood specimen and always within days of the collection. Cumulative disease damage was assessed with the Systemic Lupus International Cooperating Clinics (SLICC) Damage Index at the same time. Thirty eight normal control sera were obtained from the University of Alabama at Birmingham Blood Bank. Forty sera and 40 synovial fluids from patients fulfilling the ACR criteria for rheumatoid arthritis were also collected and stored as above. All studies were reviewed and approved by the Institutional Review Board.

ELISA for measurement of BLyS:

The sandwich ELISA for measurement of the soluble form of BLyS was developed in Human Genomic Sciences, Inc. Briefly, 96 well plates were coated with purified monoclonal anti-BLyS antibody (clone: 15C10) at 3 μg/ml in PBS at 4° C. over night, then blocked with 1% BSA PBS. The purified recombinant BLyS was used as standard. All sera or plasmas were pre-absorbed with protein A-agarose to deplete Igs and 1:10 diluted with 3% BSA PBS and incubated in the ELISA plate at 4° C. over night. After washing, the plate was further incubated with 0.2 μ/ml biotin conjugated polyclonal anti-BLyS antibody at room temperature for 2 hours. After additional washing, the plate was incubated with 1:30,000 diluted HRP-conjugated streptavidin (Southern Biotechnology, Birmingham, Ala.) for an additional one hour at room temperature. The reaction was developed by the TMB substrate (Sigma, St. Louis, Mo.) and read in an E-Max plate reader (Molecular Device, Sunnyvale, Calif.). A standard curve was made and absolute values were calculated.

Immunoprecipitation and Western blot analysis of BLyS:

Purified monoclonal anti-BLyS antibody (15C10) was conjugated to CNBr activated sepharose beads (Pharmacia, Uppsala, Sweden) according to the manufacturer's instruction. The recombinant BLyS was serially diluted in 3% BSA PBS as control. One ml of each serum was pre-incubated with 100 μl of the protein A-agarose beads at room temperature for one hour. The absorbed sera were incubated with 100 μl of anti-BLyS conjugated beads at 4° C. overnight. The beads were washed with PBS containing 0.1% Tween 20 for five times, and denatured in 50 μl of SDS loaded buffer. The samples were separated in 15% SDS-PAGE and blotted onto nylon membranes. After blocking with 5% nonfat dry milk, the blots were probed with 1 μg/ml of a second monoclonal anti-BLyS antibody (clone: 9B6) at 4° C. overnight. After washing, the blots were further incubated with HRP-conjugated goat anti-mouse IgG1 at room temperature for one hour. The blots were developed with chemiluminescence (KPL, Gaithersburg, Md.).

Assay for B cell stimulatory activity: Flat-bottom 96 well culture plates were coated with 10 μg/ml of anti-BLyS antibody at 4° C. overnight. After blocking with 3% BSA PBS, 200 μl sera were added to each of three wells and incubated for one hour at 37° C. To insure the maximum binding of BLyS to the plates, the incubation with fresh sera was repeated three times. BSA buffer was used as a control The splenic B cells of Balb/c mice were used as the indicator of B cell proliferation. The B cells were enriched from spleen by using anti-thyl0.2 antibody and complement to deplete Thy1.2 positive cells. The 5×10⁵ B cells were added and incubated with 2 μg/ml of F(ab)₂ anti-μ-polyclonal antibody (Jackson ImmunoResearch). The cultures were carried out for 72 hours, and B cell proliferation was determined by ³H thymidine incorporation assay. The proliferation index is presented as the ratio of cpm in the presence of sera and in the absence of sera.

Assay for autoantibodies and total immunoglobulins:

ELISA kits for anti-dsDNA, anti-Sm and SmRNP were purchased from Helix Diagnostics (West Sacramento, Calif.). Assays were performed according to the manufacturer's instructions except that HRP-conjugated anti-human IgM and IgA (Souther Biotechnology) were used for Ig classes. Total IgG, IgM and IgA were measured by ELISA; the paired, purified and HRP-conjugated anti-human IgG, IgA and IgM were purchased from Souther biotechnology, and affinity-purified human IgG, IgM and IgA were used as standards.

Statistical:

Statistical analysis was performed using the Student t test for comparison of population samples. Value of p<0.05 was used to reject the null hypothesis.

When measuring two independent sets of SLE sera (SLE1) and plasma (SLE2) using the ELISA, it was found that serum levels of BLyS in both sets of samples from SLE patients were significantly higher (p<0.0001) than in normal controls. (See FIG. 1. Note the horizontal bars which indicate the average of each group.) A very similar pattern of BLyS was found in both patient populations. The serum BLyS levels in the majority of normal controls were below 5 ng/ml, and less than 10% were higher than 10 ng/ml. None of the normal controls was above 12 ng/ml. In contrast, the BLyS levels in most SLE patients were higher than 5 ng/ml, and more than 30% were above 10 ng/ml. Approximately 10% of SLE patients exhibited very high levels (>20 ng/ml) of BLyS. The BLyS levels in a few SLE patients were as high as nearly 40 ng/ml.

BLyS can naturally exist in both membrane-bound and soluble forms. To determine whether the BLyS in sera corresponds to the predicted soluble form of BLyS, immunoprecipitation with an anti-BLyS monoclonal antibody was performed. Using the recombinant soluble BLyS as control, immunoprecipitation of BLyS revealed a 19 kD protein. Dose-dependent immunoprecipitation of recombinant BLyS demonstrated a threshold for detection of about 15 ng/ml. The soluble form of BLyS was detected in all 16 sera of SLE patients tested, and the size of the immunoprecipitated BLyS exactly matched the 19 kD of the recombinant BLyS. However, only one of eight normal controls weakly showed a match. Taken together, these results indicate that serum levels of BLyS are elevated in the patients with SLE, and the increased BLyS in SLE sera exists in the soluble form, which is cleaved from the cell surface.

To determine whether the BLyS in the sera of SLE patients is functional, the B cell co-stimulation assay was performed using an anti-BLyS monoclonal antibody to capture the BLyS in serum onto 96 well plates and then to co-stimulate B cells in the presence of anti-μ antibodies. In the co-culture of anti-μ antibody-stimulated B cells with the antibody captured recombinant BLyS, a dose-dependent B cell proliferative response was observed. A significantly increased B cell proliferation response was seen in the presence of ≧10 ng/ml of the antibody-captured recombinant BLyS, indicating that this method is able to detect the functional BLyS. The B cell co-stimulatory activity captured by anti-BLyS antibody in the sera of SLE patients was significantly higher (−<0.001) than that of normal controls (FIG. 3b). While normal sera showed no significant co-stimulatory activity, most sera from SLE patients exhibited increased co-stimulatory activity in anti-μ induced B cell proliferation. The B cell co-stimulatory activity captured in SLE sera is specific for BLyS, because the pre-absorption of SLE sera with anti-BLyS antibody eliminated the activity. These results indicate that BLyS is not only increased in SLE patients, but also can function as a B cell stimulator.

Study relating to Sjogren's syndrome:

A study was conducted to evaluate the role of BLyS in Sjogren's syndrome (SS). Sera from patients with SS according to revised European criteria (4 criteria with focus score ≧1 or presence of anti-SSA/SSB antibodies) were studied. The absolute value of BLyS was determined using the ELISA assay as previously described. Correlations between BLyS and the presence of anti-SSA/SSB antibodies, rheumatoid factor and the level of gammaglobulin were determined. Statistical analysis was performed using Mann-Witney and ANOVA tests. Forty-nine patients were included in the study.

Results showed serum BLyS level was increased in SS patients compared to controls: 8.58 ng/ml versus 2.56 ng.ml (p<0.0001). The level of BLyS was associated with the presence of anti-SSA/SSB: 10.45 ng/ml versus 5.60 ng/ml (p=0.008). the presence of rheumatoid factor was 10.76 ng/ml versus 6.30 ng/ml (p=0.03). When three patients with a monclonal component were excluded, the same comparisons remained positive. An association was also present relating to the level of gammaglobulins (p=0.004) and the level of IgG (p=0.02).

The finding that BLyS level is associated with anti-dsDNA antibody, but not other anti-nuclear protein autoantibodies, indicates that the mechanism for production of antibodies against dsDNA might be different from that of other autoantibodies against nuclear proteins. Because, BLyS can play a crucial role in early activation of self-antigen-driven autoimmune B cells with autoimmune T cells, further driving the switch for production of pathogenic IgG autoantibodies, the measurement of BLyS can indicate the presence of disease and can serve as an evaluative tool in management of disease conditions wherein anti-dsDNA is a factor in pathology of the disease.

It is noted that patients with a positive ANA, but no other ACR criteria for lupus, had marginally elevated BLyS levels while those with a positive ANA and several criteria for lupus had even higher levels, suggesting that an elevated BLyS precedes the formal fulfillment of criteria for SLE and can be useful as a marker for early activation of an autoimmune diathesis.

BLyS naturally exists in both membrane-bound and soluble forms and may be produced primarily by monocytes. The data from the studies disclosed herein indicate that the protein form of circulating BLyS in patients such as those studied in the examples is consistent with the naturally cleaved, soluble form and that BLyS can function as a potent B cell stimulator comparable to recombinant soluble BLyS.

In order to determine whether increased levels of BLyS play a role in the production of autoantibodies, the sera of SLE patients were divided into two groups according to their BLyS levels: SLE^{hi}, (BLyS>15 ng/ml) and SLE^{lo} (BLyS<5 ng/ml) (Table 1). The SLE patients with high levels of BLyS exhibited significantly higher levels of anti-dsDNA antibody in each of the IgG, IgM and IgA classes compared to the SLE patients with low levels of BLyS and normal controls (p<0.0001). The percentage of positive anti-dsDNA antibody was also significantly higher in the patient group with hi BLyS, with anti-dsDNA antibody IgG, IgM and IgA positive at the level of 80%, 80% and 60%, respectively, for the SLE^{hi} compared to 30%, 20% and 10% for the SLE^{lo} group. Total IgA levels were slightly, but significantly (p<0.005) higher in the SLE^{hi} group compared to the SLE^{lo} and control groups, while total IgG and IgM levels showed no differences. Two major anti-nuclear protein autoantibodies, anti-Sm and anti-SmRNP, were also measured in two SLE and control groups. Both autoantibody levels were significantly higher in both SLE groups compared to normal controls, but there was no consistent difference between the BLyS high and BLyS low groups (data not shown). These results indicated that increased levels of BLyS in patients studied are associated with increased production of anti-dsDNA antibodies, which may participate in disease pathogenesis, but not with other anti-nuclear protein antibodies.

TABLE 1
Correlation of high BLyS levels with anti-dsDNA antibody

| | | SLE | |
|---|---|---|---|
| | Normal | BLyS^{hi} | BLyS^{lo} |
| Number | 10 | 10 | 10 |
| BLyS | 4.87 ± 0.99 | 20.77 ± 2.37*** | 4.61 ± 0.51 |
| anti-dsDNA- | 0.108 ± 0.017 | 1.165 ± 0.427*** | 0.242 ± 0.080 |
| IgG | (0/10) | (8/10) | (3/10) |
| anti-dsDNA- | 0.068 ± 0.008 | 0.712 ± 0.292 | 0.104 ± 0.016 |
| IgM | (0/10) | (8/10) | (2/10) |
| anti-dsDNA- | 0.055 ± 0.005 | 0./170 ± 0.038 | 0.060 ± 0.009 |
| IgA | (0/10) | (6/10) | (1/10) |
| total IgG (μg/ | 1898 ± 188 | 2560 ± 221 | 1729 ± 183 |
| ml) | | | |
| total IgM (μg/ | 182 ± 21 | 193 ± 30 | 206 ± 20 |
| ml) | | | |
| total IgA (μg/ | 437 ± 85 | 693 ± 138 | 610 ± 47 |
| ml) | | | |

Note that the SLE samples were divided into BLyS^{hi} and BLyS^{lo} groups and compared to normal controls. Anti-dsDNA antibody and total IgG, IgM and IgA were determined by ELISA. The results are presented as mean ± SEM. The positive value was determined by the mean ± SD of normal control. The p value was determined by t-test between BLyS^{hi} and BLyS^{lo} groups.
***p < 0.0001;
**p < 0.005.

Although BLyS levels were associated with anti-dsDNA antibody, they were not associated with global organ damage as determined by the SLICC index or with renal damage as determined by serum creatinine. This would indicate that single point increases in the levels of BLyS are unlikely markers for the activity and severity of SLE. However, data from two subgroups of the patients with positive antinuclear antibodies (ANA) who did not meet the formal ACR criteria for classification of SLE was collected and analyzed. The first group with only a positive ANA exhibited slightly higher BLyS levels (8.59±0.82, n=8) was compared to normal controls. In contrast, the second group with a positive ANA and the clinical impression of lupus had significantly increased BLyS levels (14.94±2.99, n=5) (p<0.01 between two groups). These results indicate that an elevated level of BLyS precedes the formal fulfillment of the criteria and is appropriate for evaluating immune activation.

The monitoring of changes in BLyS in the circulation may be used as a means of evaluating SLE and other autoimmune disease activity. More generally, the monitoring of changes in BLyS level can be used as a means of following B cell burden/activity in the patient. Additionally, it may be helpful to periodically evaluate BLyS levels in patients who have recently suffered from infections which are sometimes precursors to autoimmune disease, such as streptococcal infections.

The use of antibodies or blocking peptides to counteract increased levels of BLyS in patients with elevated BLyS may be used as a treatment to control untoward effects of BLyS in patients suffering from self-antigen driven autoimmune disease. The amount administered should be sufficient to block circulating BLyS. The BLyS activity inhibiting amount of antibody or blocking peptide used would depend on the condition, size and age of the patient as well as the amount of circulating BLyS. The antibodies or blocking peptides may be delivered parenterally by, for example, intravenous injection or directly to the adversely affected tissue. The antibodies to BLyS may be formulated in liposomes for administration to the patient.

What we claim is:

1. A method of treating a patient with autoimmune disease having an elevated BLyS by administration of a composition containing an amount of antibody to BlyS sufficient to bind excess BLyS to a patient in need thereof.

2. The method of claim 1 wherein the patient has systemic lupus erythematosus.

3. The method of claim 1 wherein the anti-BLyS antibody is provided in a liposome.

4. A method of monitoring the changes in condition of a patient displaying symptoms of autoimmune disease comprising periodic evaluation of the level of BLyS in the blood of said patient using BLyS-binding antibodies by:
1) exposing samples of (a) sera or plasma from patients suffering from diseases arising from self-antigen driven autoimmune response to anti-BLyS antibody and (b) at least one control sample containing a known amount of BLyS to anti-BLyS antibodies,
2) incubating the product of step 1 for a time sufficient to allow said anti-BLyS antibody to bind to BLyS in the samples,
3) evaluating the amount of anti-BLyS antibody bound to BLyS in each of the patient and control samples, and
4) comparing the amount of BLyS in control samples patient samples to determine amount of BLyS in the patient samples.

5. The method of claim 4 wherein the test is an ELISA test.

6. The method of claim 4 wherein, in step 1, the anti-BLyS antibody is on a solid support.

7. The method of claim 6 wherein the support is a microtiter plate.

8. The method of claim 6 wherein the solid support is sepharose beads.

9. The method of claim 4 which is a Western blot test.
